(19) United States
(12) Patent Application Publication
(10) Pub. No.: US 2014/0074148 A1
(43) Pub. Date: Mar. 13, 2014

(54) EMBOLIC PROTECTION SYSTEM AND METHOD FOR USE IN AN AORTIC ARCH

(71) Applicant: Synecor LLC, Chapel Hill, NC (US)

(72) Inventors: Richard A. Glenn, Chapel Hill, NC (US); Michael S. Williams, Santa Rosa, CA (US); Kevin Holbrook, San Jose, CA (US); Grant Williams, Santa Rosa, CA (US); Kris Kristoffersen, Redding, CA (US)

(73) Assignee: Synecor LLC, Chapel Hill, NC (US)

(21) Appl. No.: 13/773,625

(22) Filed: Feb. 21, 2013

Related U.S. Application Data
(60) Provisional application No. 61/601,555, filed on Feb. 21, 2012.
(60) Provisional application No. 61/611,535, filed on Mar. 15, 2012.

(51) Int. Cl. A61F 2/01; A61F 2/24

(52) U.S. Cl. CPC A61F 2/013; A61F 2/24
USPC 606/200

(57) ABSTRACT

A device temporarily positionable within an aortic arch for deflecting embolic particles released during a therapeutic or diagnostic procedure comprises a resilient frame defining an opening and a barrier disposed in the opening. The barrier has a plurality of openings, which may be pores, proportioned to allow passage of blood therethrough but to prevent passage of embolic particles. The barrier has a concave shape having a convex surface positionable in contact with a wall of an aorta to cover at least a brachiocephalic ostium. The frame may include an upstream end including a pair of lobes and an apex between the lobes, and has a generally tapered downstream end.

This application claims the benefit of U.S. Provisional Application No. 61/601,555, file Feb. 21, 2012, and U.S. Provisional Application No. 61/611,539, filed Mar. 15, 2012, each of which is incorporated herein by reference.

### TECHNICAL FIELD OF THE INVENTION

The present invention relates to the field of devices and methods for preventing embolic particles from entering the cerebral vascular during cardiac procedures and/or intravascular medical procedures.

### BACKGROUND

Various medical procedures performed using devices placed in the heart and/or through the vasculature can result in the release of embolic material into the blood. Release of embolic material can occur, for example, through dislodgement of plaque from diseased vessels or valves and/or the release of thromboembolic material. Examples of such procedures include, for example, transcatheter aortic valve implantation procedures (“TAVI”).

Devices and methods disclosed herein improve upon the safety of such medical procedures by diverting embolic material released during such procedures away from the major arteries feeding the brain, particularly the carotid arteries. In particular, a temporary device is positioned to divert emboli away from branches in the aortic arch.

### BRIEF DESCRIPTION OF THE DRAWINGS

FIG. 1A schematically shows a first embodiment of a diverter disposed in an aortic arch.

FIG. 1B is similar to FIG. 1A but shows an alternate diverter.

FIG. 1C shows the diverter of FIG. 1B with the longitudinal struts visible.

FIG. 2A schematically shows a second embodiment of a diverter disposed in an aortic arch.

FIG. 2B is a lateral view of the diverter of FIG. 2A.

FIG. 2C is a superior view of the diverter of FIG. 2A.

FIG. 2D is similar to FIG. 2A but illustrates deflection of particles.

FIGS. 2E and 2F are a lateral view and a superior view, respectively, showing the diverter of FIG. 2A positioned in an aortic arch.

FIGS. 2G and 2H illustrate deployment of the diverter of FIG. 2A.

FIGS. 3A through 3D illustrate positioning of a third embodiment of a diverter within an aortic arch.

FIG. 3E is similar to FIG. 3A and illustrates positioning of other instruments within the aortic arch.

FIG. 3F illustrates an embodiment of the FIG. 3A embodiment modified for transaortic use.

FIG. 3G schematically illustrates positioning of the FIG. 3F embodiment.

FIG. 4 schematically illustrates positioning of a filter device for capturing diverted particles.

### DETAILED DESCRIPTION

### First Embodiment

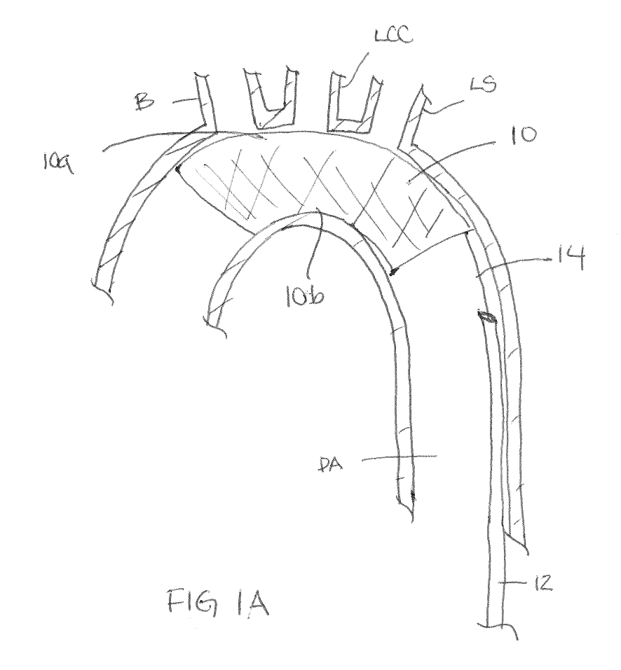

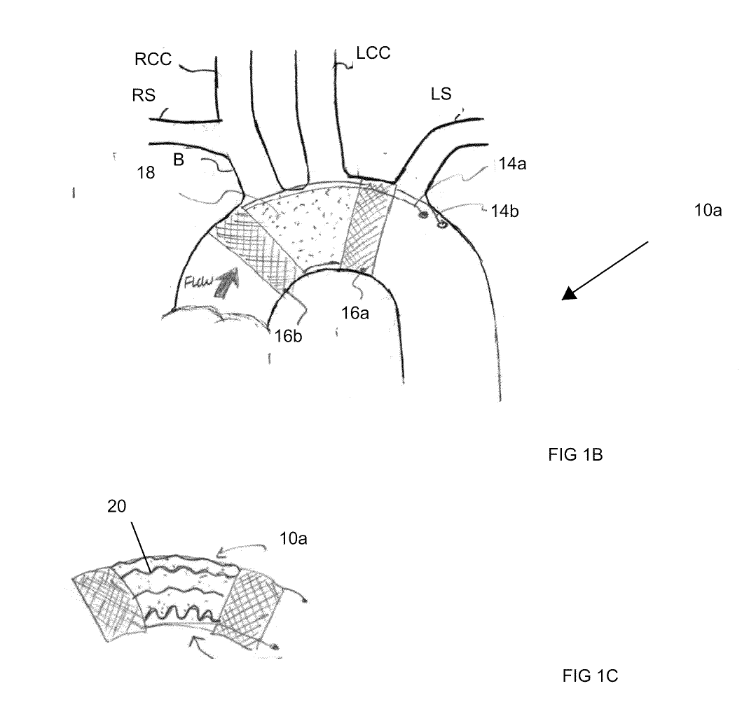

FIG. 1A schematically shows a first embodiment of an embolic diverter 10 disposed within an aortic arch. In this embodiment, the diverter 10 is formed as a self-expanding tubular device formed of braid or mesh. The diverter has sufficient length to cover the ostium of the brachiocephalic artery (through which blood flows into the right subclavian and right common carotid arteries) and the left common carotid artery. In other embodiments, the length may be sufficient to also cover the ostium of the left subclavian artery as shown.

In one embodiment, the diverter 10 is dimensioned to have a uniform inner diameter when in a generally straight configuration. The diverter walls have interstices or porosity that will allow the passage of blood through the diverter walls from the aortic arch into the aortic branch vessels, but that will not allow emboli to pass through the diverter walls into the branch vessels. In one embodiment, the braid or mesh is generally uniform along the length of the diverter that bridges the branch vessels, as well as around its circumference. When deployed in the aortic arch, the portion 10a of the diverter extending along the ostia of the branch vessels will be in tension, thus slightly enlarging the pores or interstices in the diverter 10, thus increasing its porosity along the ostia. The portion 10b of the diverter wall lining the shorter-radius curve the aorta will be slightly compressed.

The diverter 10 may be provided with or without a porous membrane covering the inner and/or surface of the braid, or otherwise coating the braid.

In use, the diverter 10 is disposed within a catheter 12 and introduced into the vasculature through an access port in the femoral artery. The distal end of the catheter 12 is advanced through the descending aorta and positioned with its distal opening upstream of the brachocephalic artery B. The catheter 12 is withdrawn from the diverter 10 using techniques known in the art. When released from the catheter, the diverter expands into contact with the aortic arch with its distal end disposed upstream of the ostium of the brachiocephalic artery B and with its proximal end disposed downstream of the ostium of the left common carotid artery LCC or left subclavian artery LS. The tether 14 is coupled to the proximal end of the diverter 10 for use in removing the diverter 10 following a procedure.

In its expanded state, the braid preferably lies in firm contact with the surrounding walls of the aortic arch so as to avoid obstructing blood flow through the lumen of the arch—and the walls of the braid cover the ostia of the brachiocephalic and left common carotid arteries. Once the braid has been positioned, a procedure is performed in the heart or vasculature. For example, a TAVI procedure may be performed using instruments that are introduced into the descending aorta DA via a femoral approach and then passed through the lumen of the diverter to the aortic valve. Emboli that are released during the procedure and that pass into the ascending aorta cannot pass into the brachocephalic and left common carotid arteries due to the presence of the diverter walls covering the entrances to those arteries. The embolic material thus bypasses the ostia of the covered vessels and exits the aortic arch through the descending aorta.

Once the procedure has been completed, the tether 14 is engaged using a snare or other instrument. Tension is applied to the tether 14 while the catheter 12 is advanced distally, causing the braid to collapse collapsible to the catheter 12. As the diverter is collapsed, embolic particles remaining in the diverter's lumen are drawn into the retrieval catheter with the diverter. The catheter 12 is then withdrawn from the vasculature.

In a slightly modified embodiment shown in FIG. 1B, diverter 10a includes proximal and distal anchoring sections 16a,b which may be formed of mesh, braid, or another self-expandable structure capable of expanding into contact with the walls of the aortic arch. A porous member 18 extends between the anchoring sections such that it is positioned covering the ostia of the brachiocephalic and left common carotid arteries, and optionally the left subclavian artery. As shown in FIG. 1C, longitudinal struts 20 (which may have an undulating shape that at least partially straightens and lengthens in response to tension and compresses and shortens under tension) may extend between the anchoring sections 16a,b. The struts 20 aid in providing stability to the anchoring sections 16a,b.

In the FIG. 1B embodiment, each of the anchoring sections 16a, 16b may have a separate tether 14a, 14b to facilitate withdrawal.

In a variation of the FIG. 1B embodiment, the membrane 18 does not extend the full circumference of the aortic arch, but covers enough of the aortic wall to cover the desired ostia and the surrounding tissue. In this variation, the diverter may be provided with markers to aid in radial positioning of the diverter under fluoroscopy.

### Second Embodiment

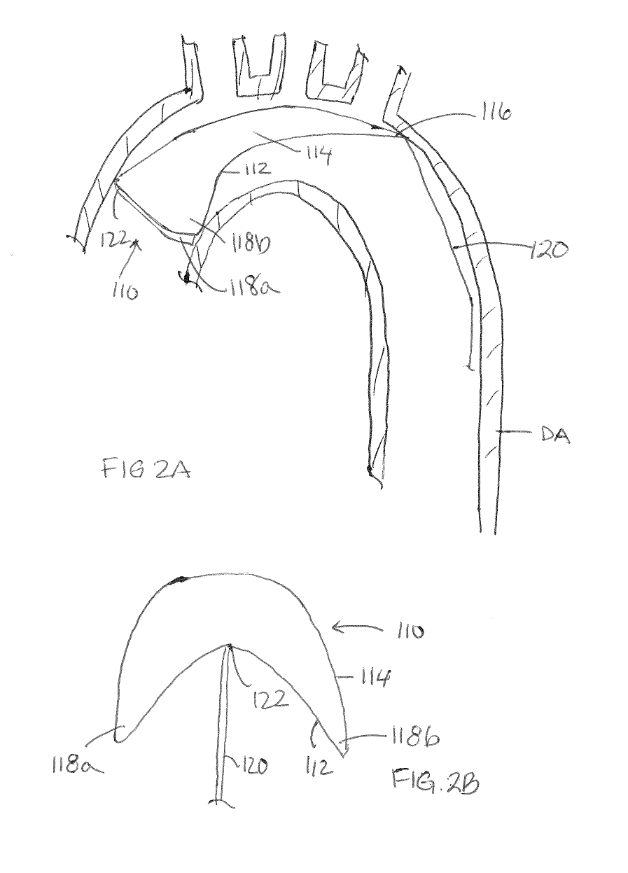

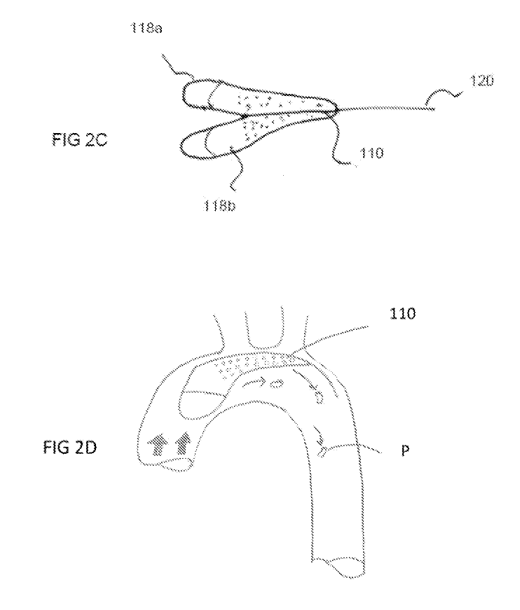

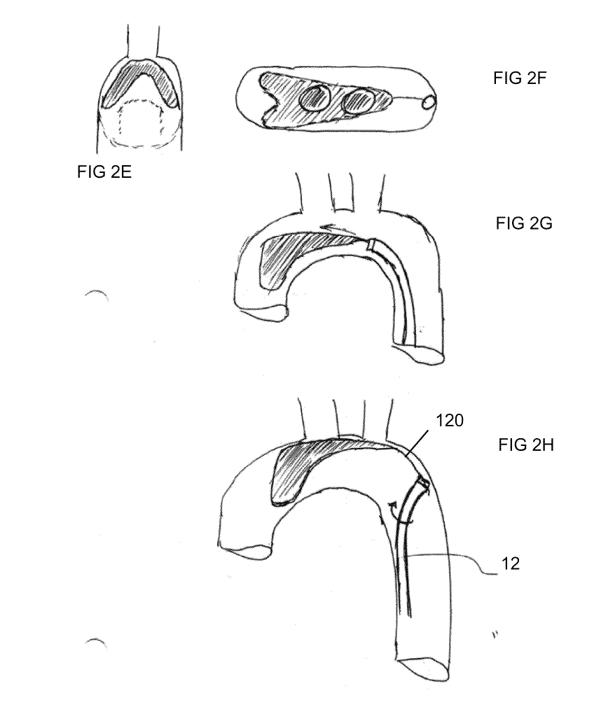

The diverter 110 of the second embodiment, shown in FIGS. 2A through 2J, takes the form of a shield positionable over the target ostia (i.e. the ostia of the brachiocephalic and left common carotid arteries, and optionally that of the left subclavian artery).

This diverter is formed of a flexible frame 112 defining an open area. A barrier 114 is supported by the perimeter of the frame. The barrier is one that will prevent passage of emboli through the frame, but at least certain regions of the barrier are porous so as to allow allowing blood to flow through it. In one embodiment, the porous barrier may be formed of porous silicone or polyurethane, or other materials such as woven materials. In one embodiment, the covering may be applied using dip, molding and/or spray techniques. The barrier preferably contacts the full inner perimeter of the frame, but in some embodiments the outer perimeter of the frame may be formed to be free of the barrier material to facilitate sliding of the diverter within the delivery and removal catheter(s) 12 (FIG. 1A).

The frame is preferably made of nitinol or similar material, and is shape set to the desired shape. Referring to FIG. 2A, the diverter includes a proximal end 116 forming a generally V- or U-shaped tail section extending towards the descending aorta DA (FIG. 2A). The frame defines a pair of lobes 118a,b at the distal end (towards the ascending aorta—on the left side of FIG. 2A). The lobes meet at a generally U- or V-shaped apex 122, which is longitudinally aligned with the apex of the proximal tail section.

As shown in FIG. 2C, when viewed from the top (e.g. cranial view) a longitudinal axis extends between the proximal tail section and the apex between the lobes. The lobes extend laterally from this longitudinal axis, giving the diverter and frame an approximate heart-shaped appearance. In one embodiment, the perimeter of the frame may have a proximal portion 124 that extends distally from the longitudinal axis at a first angle, and a distal portion 126 that extends at a second, greater, angle in the region distal to the apex between the lobes 118a,b. The transition from the first to the second angle is also clearly visible in FIGS. 2A and 2D.

As best seen in the lateral views (looking towards the distal end of the device as in FIGS. 2B and 2E), the lobes also curve laterally, approximating the contour of the aorta and giving the lobes a fang-type appearance when viewed laterally.

An elongate support or wire 120 extends from the proximal tail section through the descending aorta and is used to support the diverter during use. As shown in FIG. 2C, the wire may extend further distally and be coupled to the apex between the lobes to facilitate retraction of the diverter into a catheter. Although the figures show the wire as a separate element that is attached to the frame, the frame and support may be formed of one continuous wire during heat setting, thus removing the need to connect or couple to frame to the tethering wire.

As illustrated schematically in FIG. 2D, the distal portion of the barrier 114 positioned distal to the brachocephalic ostia as shown, is non-porous to allow blood flowing through the aorta to push the diverter upwardly against the ostia. The lobes 118a, 118b press outwardly or laterally against the walls of the arch as shown, and they flare radially outwardly to prevent embolic particles P from entering the branch vessels off the anterior and posterior walls of the aorta. The pores/openings in the more proximal portion of the barrier 114 allow blood to flow into the branch vessels, but are too small to allow particles to pass through. The particles thus are deflected into the descending aorta DA.

Referring to FIGS. 2G and 2H, in use the diverter 110 is disposed within a catheter 12 and introduced into the vasculature through an access port in the femoral artery, with the tethering wire extending out of the body. The distal end of the catheter is advanced through the descending aorta and positioned with its distal opening upstream of the brachocephalic artery. The diverter is deployed from the catheter and the frame expands into contact with the surrounding walls of aortic arch. Preferably, the apex 122 disposed between the lobes 118a, b is positioned upstream of the ostium of the brachiocephalic artery, and the tail section 116 of the diverter 110 is positioned downstream of the ostium of the left common carotid artery. The barrier has a concave surface (the lower surface in the drawings) facing the interior or the aorta, and a convex surface that contacts the inner wall of the aorta when positioned to cover the branch vessel ostia. The lobes of the barrier extend distally from the apex, as well as laterally, such that when deployed they curve partially around the circumference of the aorta so as to stabilize the distal end of the diverter. As shown in FIG. 2H, rotating the wire 120 or catheter 12 axially (e.g. against the lateral wall of the descending aorta) helps to push the diverter upwardly against the ostia.

As discussed, in some embodiments, the portions of the barrier defined by the lobes 118a,b are not sufficiently porous to allow blood to flow through, so that the blood passing into contact with the diverter helps to press the lobes into contact with the wall of the aorta. In one embodiment, the porous regions of the barrier are proximally positioned relative to the apex 122.

Once the diverter has been positioned, a procedure is performed in the heart or vasculature. Emboli that are released during the procedure and that pass into the ascending aorta cannot pass into the brachocephalic and left common carotid arteries due to the presence of the barrier covering the entrances to those arteries. The embolic material thus bypasses the ostia of the covered vessels and exits the aortic arch through the descending aorta.

### Third Embodiment

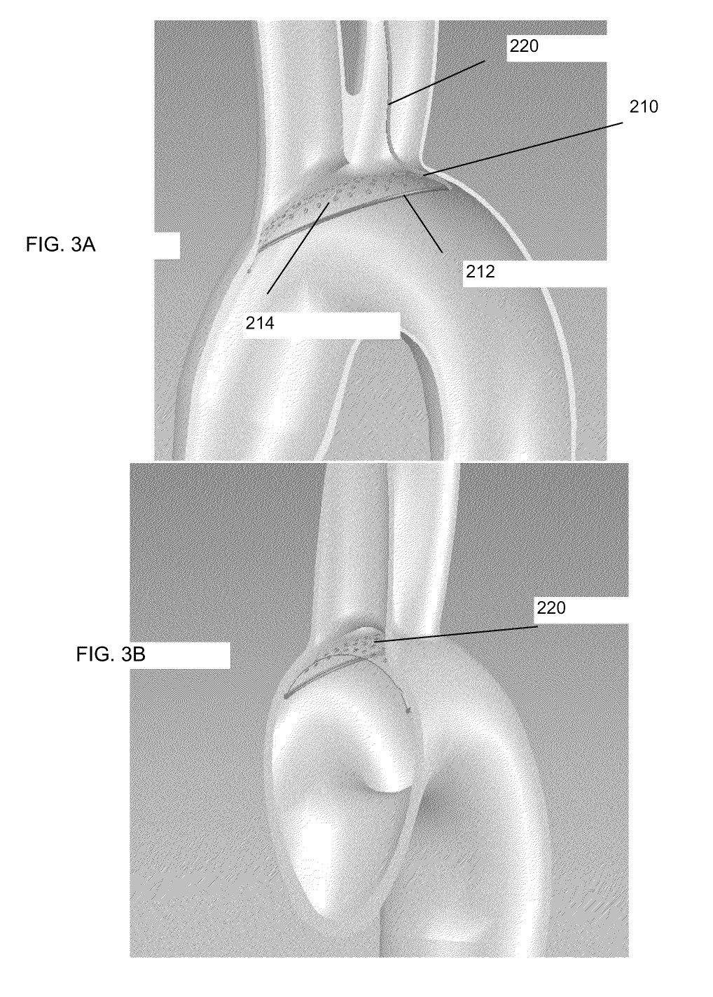

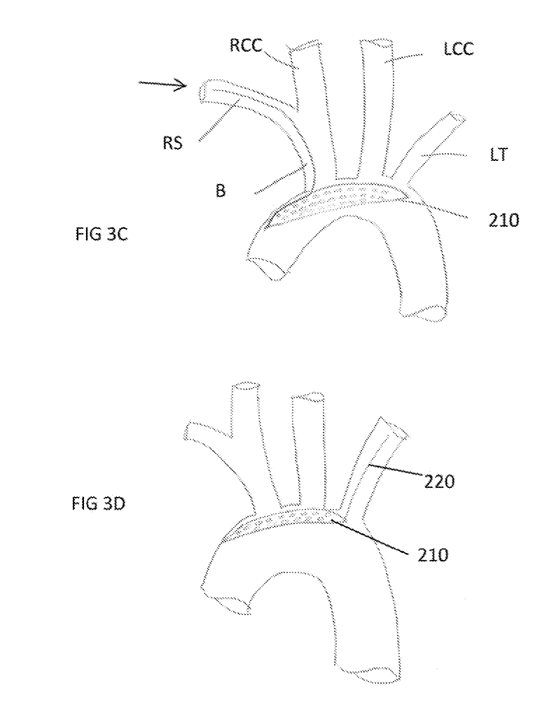

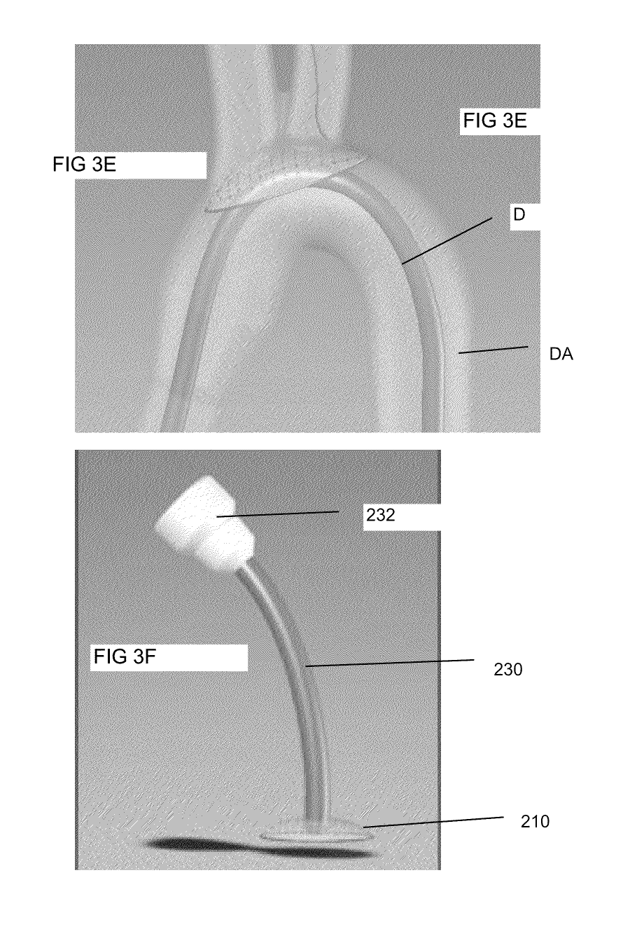

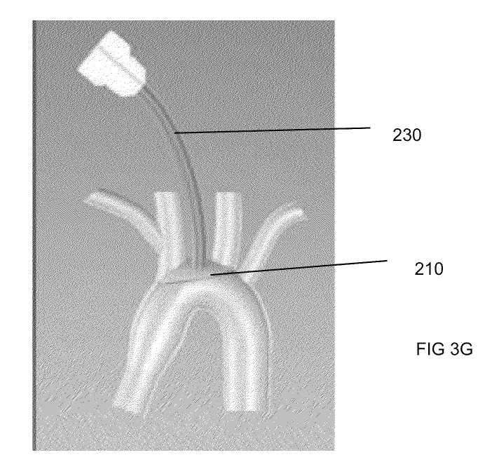

FIGS. 3A and 3B show a third embodiment of a diverter device 210. The third embodiment is similar to the second embodiment in the use of a frame 212 and a porous barrier 214 mounted to the inner perimeter of the frame. However in this embodiment, the frame and barrier define a generally oval shape. As with the second embodiment, the surface of the barrier 214 that faces into the aortic arch is concave, and the surface contacting the wall of the aorta and covering the ostia is convex—positioning the barrier away from the lumen of the aortic arch to avoid obstructing flow of blood through the arch and to further avoid advancement of instruments through the arch. For example, the arch remains free for passage of instruments passed from the descending aorta towards the aortic valve.

The wire or support 220 for this embodiment extends from the proximal end of the frame as shown. In a preferred method of using the third embodiment, the diverter is positioning using brachial access, leaving unimpeded access to the aortic valve and left ventricle for other devices D that may be introduced using a femoral approach. See FIG. 3C. Alternatively, the device is introduced through the left subclavian as shown in Fig.

In a variation of the third embodiment, the diverter is positioned at the distal end of a curved sheath 230. In use, the distal end of the sheath is introduced using a supra-aortic approach, through an incision formed in the aorta. Once the distal end of the sheath is within the aorta, the diverter is moved to the expanded position to cover the ostia of the brachiocephalic and left common carotid artery (and optionally also the ostium of the left subclavian artery). See FIG. 3E. As is common within other vascular introducer sheaths, the sheath may have a sealed/sealable port 232 on its proximal end to minimize blood loss from the aorta. Instruments used to carry out the desired procedure, e.g. a TAVI procedure, are introduced through the sheath 230. Additional instruments may optionally also be introduced femorally for use in combination with those passed through the sheath 230.

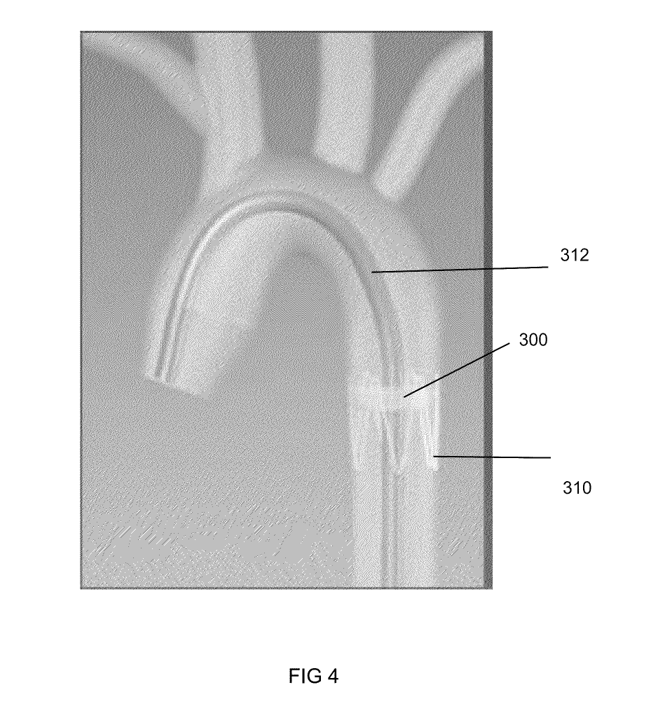

In some procedures, it may be desirable to have a proximal filter in place in the descending aorta so as to capture embolic particles deflected by the diverter. This protects the peripheral arteries by preventing the diverted particles from passing into them. In the embodiment illustrated in FIG. 4, a proximal filter 300 is supported by a stent-like structure 310 deployed from the exterior surface of an introducer sheath 312. Instruments used to carry out TAVI or other procedures may be introduced through the introducer sheath.

We claim:

1. A device for deflecting embolic particles, comprising:
a resilient frame defining an opening;
a barrier disposed in the opening, the barrier including a plurality of openings proportioned to allow passage of blood therethrough but to prevent passage of embolic particles, wherein the barrier has a concave shape having a convex surface positionable in contact with a wall of an aorta to cover at least a brachiocephalic ostium.

2. The device of claim 1, wherein the barrier has a generally oval shape.

3. The device of claim 1, wherein the frame has an upstream end including a pair of lobes and an apex between the lobes.

4. The device of claim 3, wherein the frame has a generally tapered downstream end.

5. The device of claim 3, wherein the plurality of openings in the barrier are downstream of the apex.

6. The device of claim 1, wherein the frame and barrier are positioned at a distal end of an elongate sheath, the sheath including a sealable port at its proximal end.

7. A device for deflecting embolic particles, comprising:
a resilient tubular member including a plurality of openings proportioned to allow passage of blood therethrough but to prevent passage of embolic particles, the tubular member positionable in contact with a wall of an aorta to cover at least a brachiocephalic ostium.
